(19) United States
(12) Patent Application Publication
(10) Pub. No.: US 2026/0000811 A1
(43) Pub. Date: Jan. 1, 2026

(54) ACELLULAR DERMAL MATRIX SHEET ALLOGRAFTS HAVING SPECIALIZED MESH PATTERNS

(71) Applicant: ALLOSOURCE, Centennial, CO (US)

(72) Inventors: Lauren Castillo, Castle Rock, CO (US); Ergun Kocak, Gahanna, OH (US); Kenneth Blood, Litlleton, CO (US); Jeffrey Chiesa, Denver, CO (US); Charles Manuele, Aurora, CO (US)

(21) Appl. No.: 18/877,293

(22) PCT Filed: Aug. 8, 2024
(86) PCT No.: PCT/US2024/041530

§ 371 Date: Dec. 20, 2024

Related U.S. Application Data
(60) Provisional application No. 63/531,605, filed on Aug. 9, 2023.

(51) Int. Cl. A61L 27/36; A61F 2/00; A61F 2/10

(52) U.S. Cl. CPC A61L 27/362; A61F 2/0095; A61F 2/105; A61F 2002/0081

(57) ABSTRACT

There is disclosed a packaged allograft implant configured for implantation in a human recipient. In an embodiment, the packaged allograft implant includes an acellular dermal matrix sheet having a top surface and a bottom surface in opposition to one another, a perimeter surrounding the top surface and the bottom surface, and a thickness extending between the top surface and the bottom surface. The packaged allograft implant further includes a mesh pattern extending across at least a portion of the top surface and the bottom surface of the acellular dermal matrix sheet, the mesh pattern providing through-holes extending between the top surface and the bottom surface of the acellular dermal matrix sheet, and the mesh pattern having a plurality of mesh lines extending in a first direction and a second direction. The first direction and the second direction are orthogonal to one another so as to allow a given amount of stretch in each of the first direction and the second direction. Other embodiments are also disclosed.

### BACKGROUND

Acellular dermal matrix sheet allografts may include a mesh pattern. As shown in FIGS. 1A and 1B, these prior art mesh patterns 5 are typically formed only by parallel mesh lines 10 disposed on an acellular dermal matrix sheet 20. While this mesh pattern provides various characteristics as opposed to a non-meshed acellular dermal matrix sheet, this mesh pattern only allows stretch in a single direction SD.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In an embodiment, there is provided a packaged allograft implant configured for implantation in a human recipient. The packaged allograft implant includes an acellular dermal matrix sheet having a top surface and a bottom surface in opposition to one another, a perimeter surrounding the top surface and the bottom surface, and a thickness extending between the top surface and the bottom surface. The packaged allograft implant further includes a mesh pattern extending across at least a portion of the top surface and the bottom surface of the acellular dermal matrix sheet, the mesh pattern providing through-holes extending between the top surface and the bottom surface of the acellular dermal matrix sheet, and the mesh pattern having a plurality of mesh lines extending in a first direction and a second direction. The first direction and the second direction are orthogonal to one another so as to allow a given amount of stretch in each of the first direction and the second direction.

In another embodiment, the mesh pattern is an alternating pattern of vertical and horizontal mesh lines.

In yet another embodiment, the mesh pattern is a cross pattern of intersecting mesh lines.

In still another embodiment, the mesh pattern is a diamond pattern of mirrored 45-degree angle oriented mesh lines.

In another embodiment, the mesh pattern is a multiple quadrant pattern, with parallel mesh lines in opposing quadrants and perpendicular mesh lines in neighboring quadrants, and the multiple quadrant pattern contained in a circular shaped perimeter with the mesh lines.

In yet still another embodiment, the mesh pattern is a row pattern formed by column of horizontal mesh lines followed by a column of vertical mesh lines, with the lines alternating for each column, with the mesh lines disposed on an acellular dermal matrix sheet forming an allograft for implantation in a human recipient.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIGS. 1A and 1B illustrate a prior art mesh pattern formed only by parallel mesh lines disposed on an acellular dermal matrix sheet forming an allograft for implantation in a human recipient;

FIGS. 2A-2C illustrate a novel mesh pattern formed by an alternating pattern of vertical and horizontal mesh lines disposed on an acellular dermal matrix sheet forming an allograft for implantation in a human recipient;

FIGS. 3A and 3B illustrate a novel mesh pattern formed by a cross pattern of intersecting mesh lines disposed on an acellular dermal matrix sheet forming an allograft for implantation in a human recipient;

FIGS. 4A and 4B illustrate a novel mesh pattern formed by a diamond pattern of mirrored 45-degree angle oriented mesh lines disposed on an acellular dermal matrix sheet forming an allograft for implantation in a human recipient;

FIGS. 5A-5C illustrate a novel mesh pattern formed by a harringbone pattern of repeating, alternating columns of 45-degree angle oriented mesh lines disposed on an acellular dermal matrix sheet forming an allograft for implantation in a human recipient;

FIGS. 6A and 6B illustrate a novel mesh pattern formed by a multiple quadrant pattern, with parallel mesh lines in opposing quadrants and perpendicular mesh lines in neighboring quadrants, and the pattern contained in a circular shaped perimeter with the mesh lines disposed on an acellular dermal matrix sheet forming an allograft for implantation in a human recipient;

FIGS. 7A and 7B illustrate a novel mesh pattern formed by a row pattern formed by column of horizontal mesh lines followed by a column of vertical mesh lines, with the lines alternating for each column, with the mesh lines disposed on an acellular dermal matrix sheet forming an allograft for implantation in a human recipient;

FIG. 8 illustrates a novel mesh pattern formed by a circular pattern, with arcuate mesh lines positioned about a pole location adjacent the center of the pattern, and the pattern contained in a circular shaped perimeter with the mesh lines disposed on an acellular dermal matrix sheet forming an allograft for implantation in a human recipient;

FIG. 9 illustrates a novel mesh pattern formed by alternating placement of semi-circle mesh lines, with the mesh lines disposed on an acellular dermal matrix sheet forming an allograft for implantation in a human recipient;

FIG. 10 illustrates a novel mesh pattern formed by a spiral of mesh lines, with the mesh lines disposed on an acellular dermal matrix sheet forming an allograft for implantation in a human recipient; and

FIG. 11 provides a flowchart depicting an exemplary method (200) of manufacturing an embodiment of the pre-shaped, meshed ADM graft.

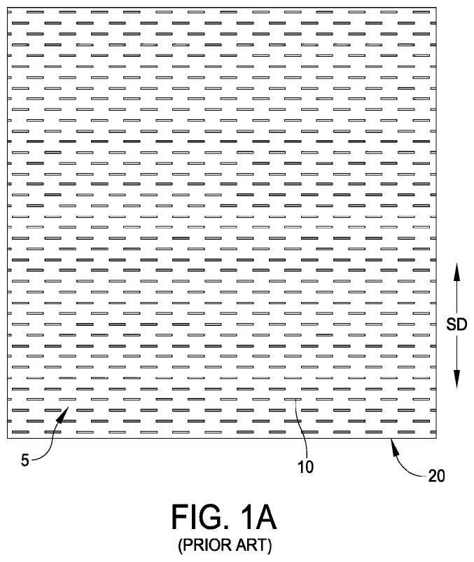

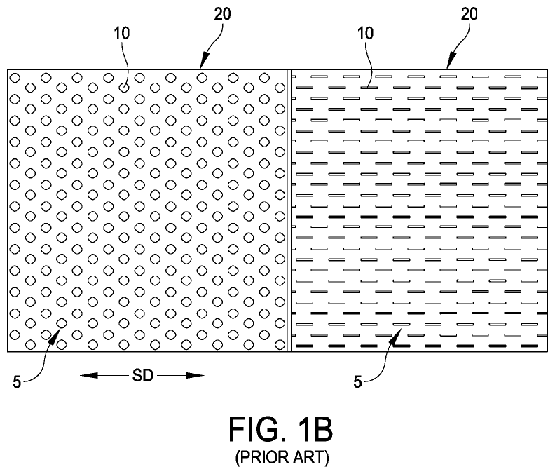

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

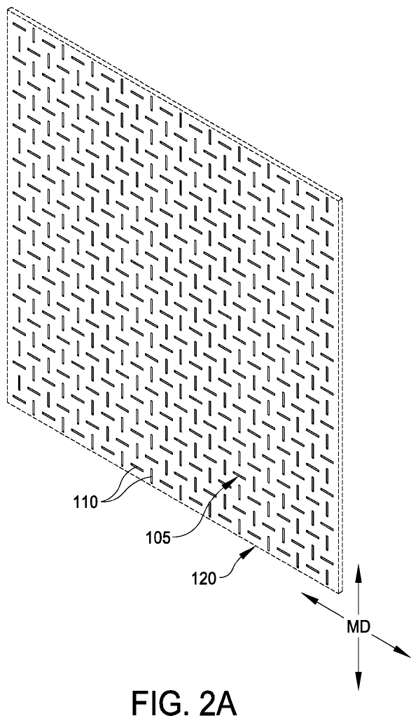

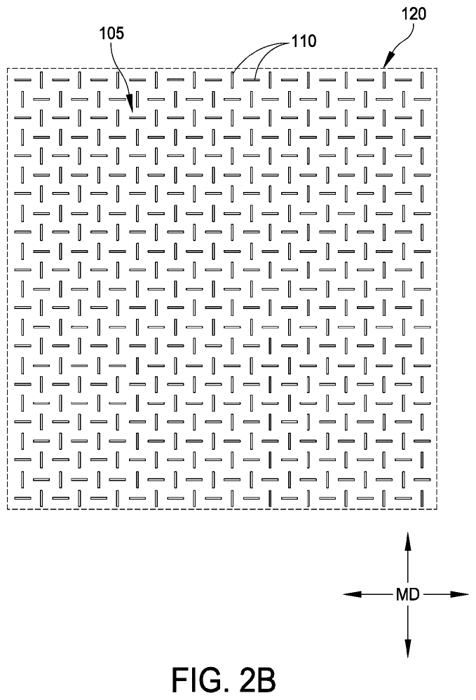

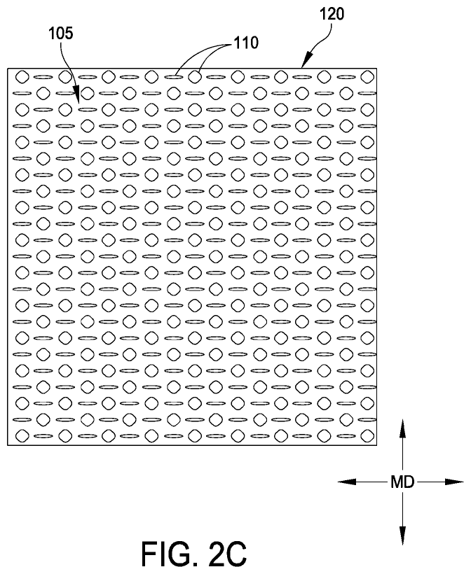

With reference to FIGS. 2A-10, and in various embodiments, there are provided novel mesh patterns 105 with mesh lines 110 in a sheet 120 for acellular dermal matrix packaged implant products which allow the product to stretch in more than one direction MD. These packaged implant products are frequently used in breast reconstruction procedures to wrap temporary expanders or permanent implants.

The mesh patterns are oriented such that the allograft can be stretched in more than one direction. The mesh lines open when stretched to allow for fluid drainage and potentially enhance integration.

In one embodiment, and with reference to FIGS. 2A-2C, there is shown a pattern of alternating vertical and horizontal mesh lines such that the allograft stretches equally in each direction. In an embodiment, the lines may be 3.48 mm long and are spaced 1.5 mm apart. In another embodiment, the lines may be 1.5 mm long and are spaced 1 mm apart. In other embodiments, the lines may be 1 mm to 4 mm long and spaced 1 mm to 4 mm apart. In various embodiments, the lines may be uniform length with respect to one another. In some embodiments, the lines may be non-uniform lengths with respect to one another. In various embodiments, the spacing may be uniform between the mesh lines. In some embodiments, the spacing may be non-uniform between the mesh lines.

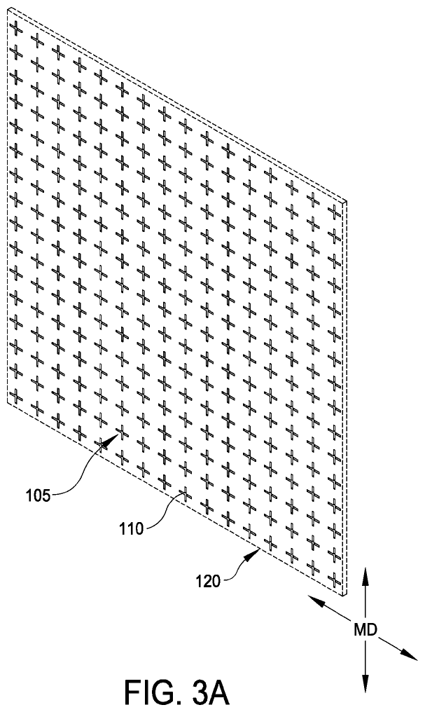

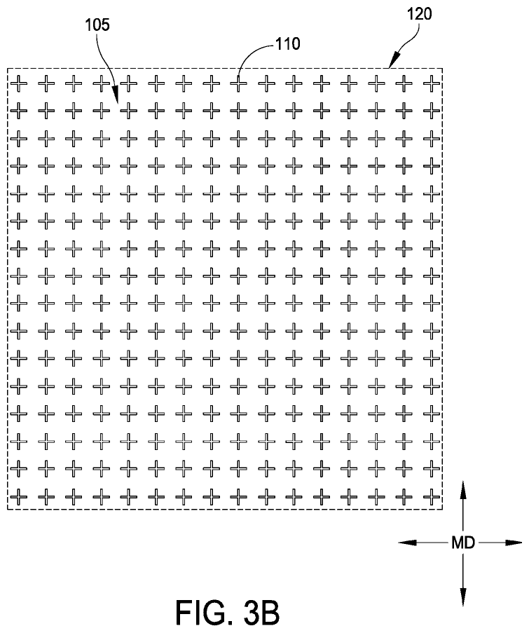

With reference to FIGS. 3A and 3B, there is shown a mesh pattern formed by a cross pattern of intersecting mesh lines disposed on an acellular dermal matrix sheet forming an allograft for implantation in a human recipient. These intersecting horizontal and vertical mesh lines form a cross.

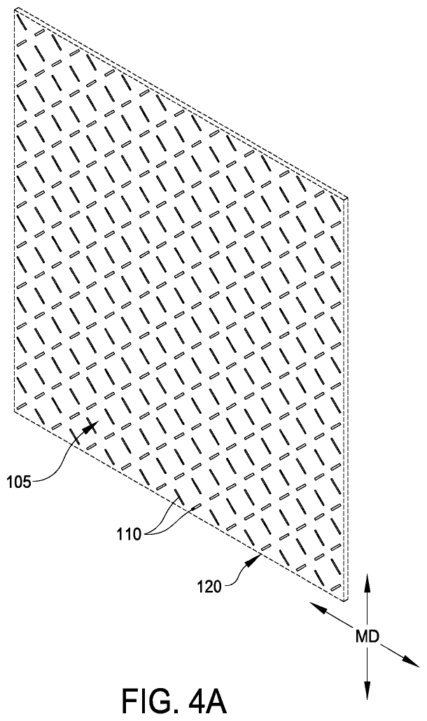

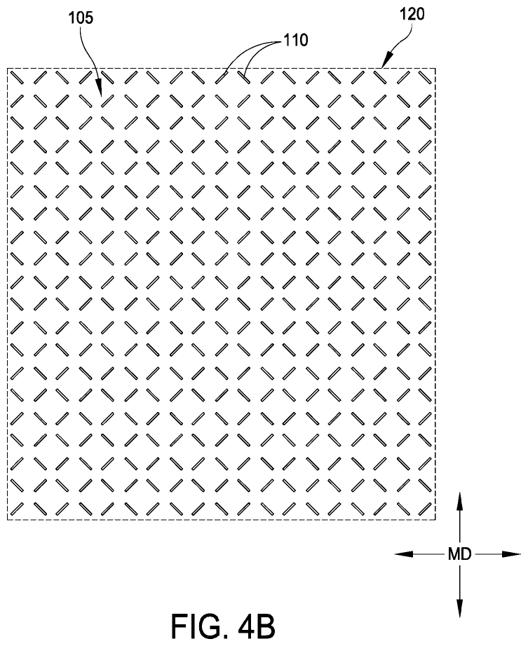

With reference to FIGS. 4A and 4B, and in another embodiment, there is shown a diamond pattern consisting of lines oriented at mirrored 45 degree angles.

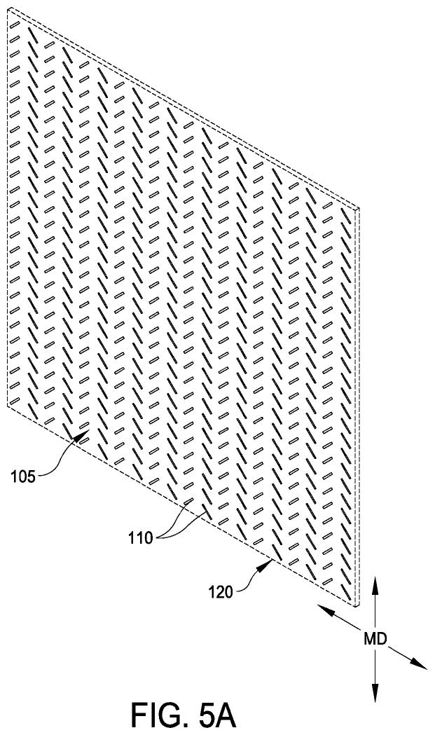

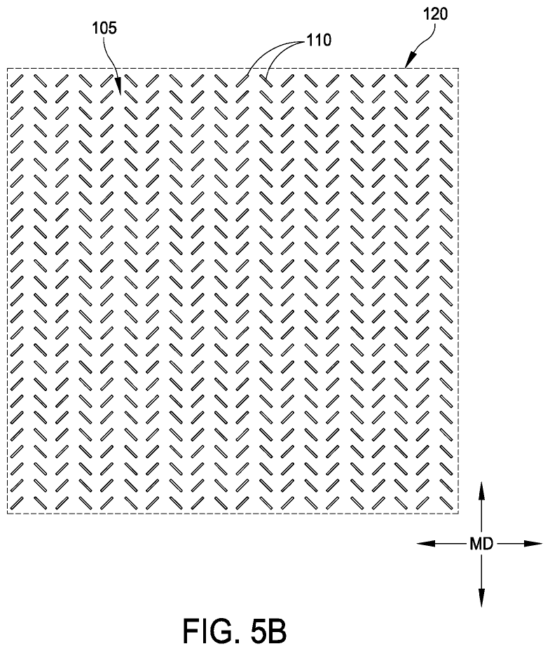

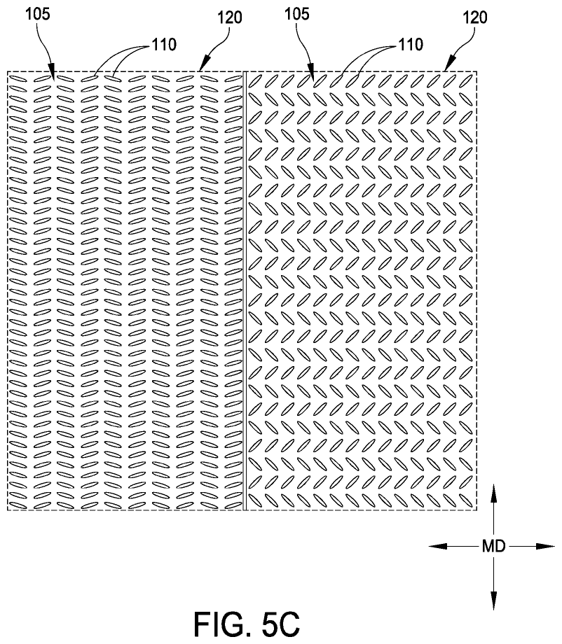

With reference now to FIG. 5, and in still another embodiment, there is shown a herringbone pattern consisting of a column of lines oriented at a 45-degree angle, followed by a column of lines oriented at the opposite 45 degree angle, repeating.

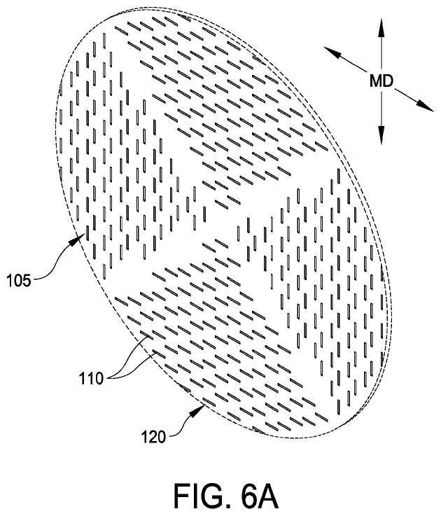

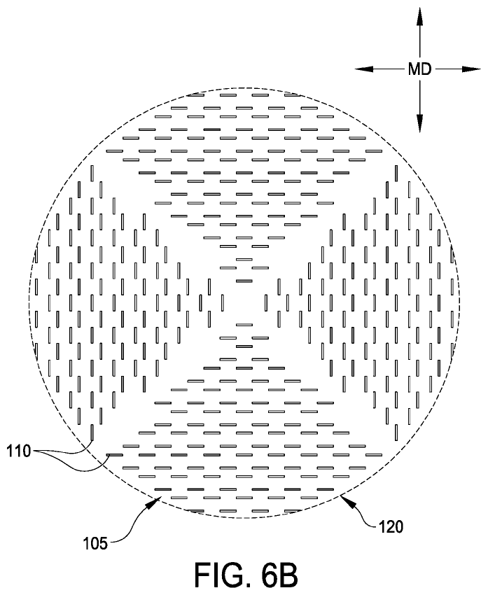

With reference to FIGS. 6A and 6B, and in an embodiment, a circular shaped graft has quadrants of parallel mesh lines where the quadrants are perpendicular to one another.

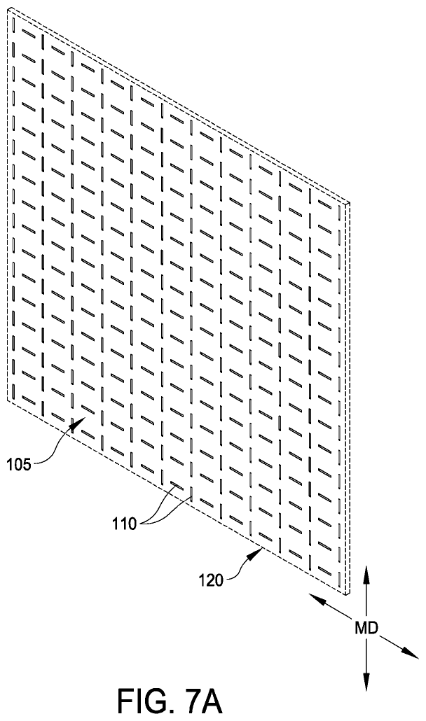

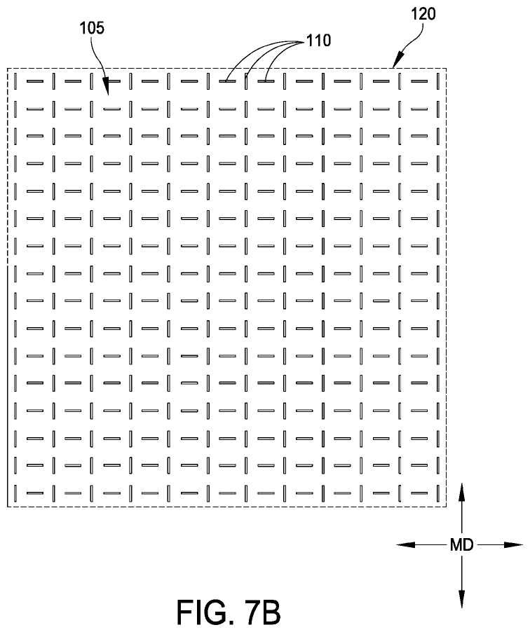

Now looking at FIGS. 7A and 7B, and in yet another embodiment, a pattern may include a column of horizontal mesh lines followed by a column of vertical mesh lines, alternating for each column.

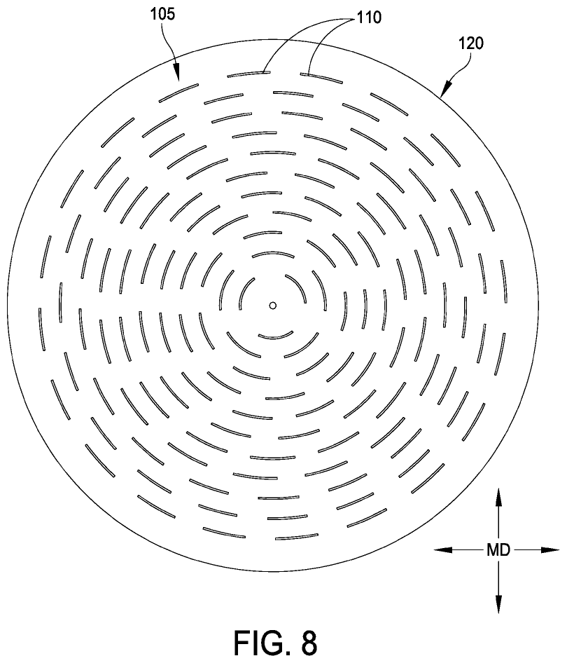

With reference to FIG. 8, and in an embodiment, a novel mesh pattern may be formed by a circular pattern, with arcuate mesh lines positioned about a pole location adjacent the center of the pattern, and the pattern contained in a circular shaped perimeter with the mesh lines disposed on an acellular dermal matrix sheet forming an allograft for implantation in a human recipient.

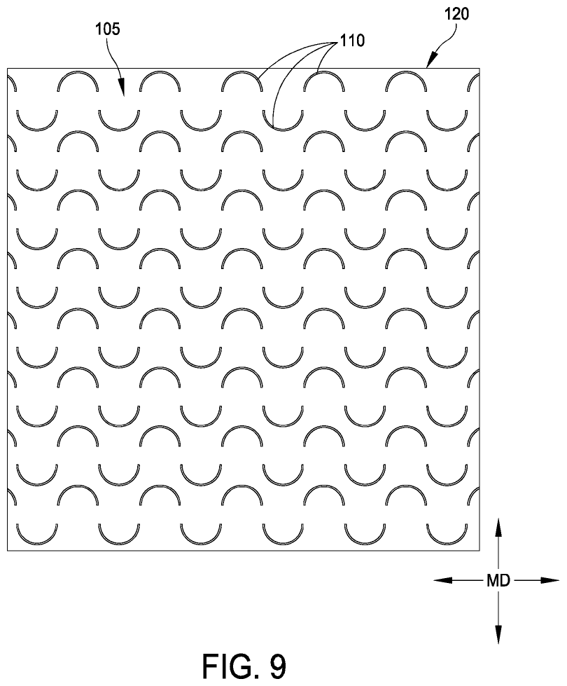

With reference to FIG. 9, and in an embodiment, a novel mesh pattern may be formed by alternating placement of semi-circle mesh lines, with the mesh lines disposed on an acellular dermal matrix sheet forming an allograft for implantation in a human recipient.

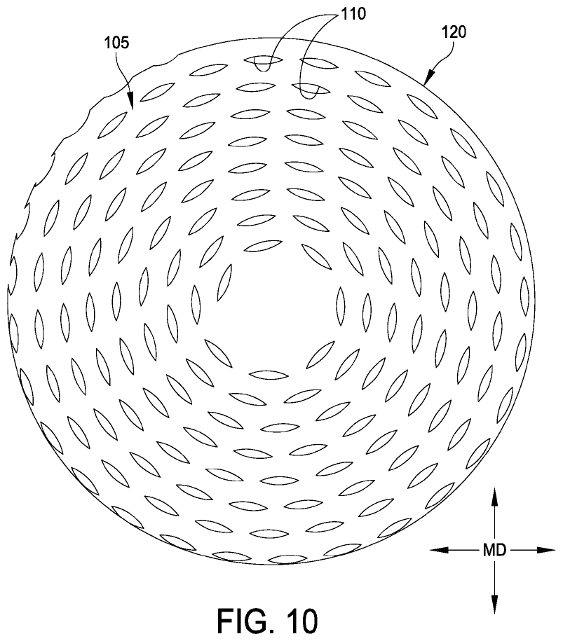

With reference to FIG. 10, and in an embodiment, a novel mesh pattern may be formed by a spiral of mesh lines, with the mesh lines disposed on an acellular dermal matrix sheet forming an allograft for implantation in a human recipient.

In various embodiments, the mesh lines may be shorter or longer than 1.5 mm and may be spaced closer or further apart.

The mesh patterns are to be used on acellular dermal matrix sheets. The mesh pattern allows the allograft sheet to be stretched in more than one direction. The sheets may be used in any application which requires stretching, fluid drainage, etc. at the surgeon's discretion (e.g., breast reconstruction implant wrapping, pelvic organ prolapse, etc.)

Current commercialized mesh patterns for skin allografts consist of only parallel straight lines. The allograft implant products of the current disclosure include mesh patterns with non-parallel lines.

Parallel mesh lines allow the skin graft to stretch significantly when pulled in the direction perpendicular to the lines but give little to no stretch when pulled in the direction parallel to the lines. When pulled parallel to the lines, the mesh is also pulled tight which does not allow for much fluid drainage. This development solves the problem by allowing the graft to stretch in more than one direction and allows the mesh pores to open regardless of the direction of stretch. The parallel mesh pattern has weaker tensile strength when pulled in the direction perpendicular to the mesh lines compared to the direction parallel to the mesh lines. The development may provide more equal strength regardless of the direction of pull.

There are commercialized skin grafts which contain curved or non-parallel fenestrations to allow for fluid drainage, but these do not allow for the same stretch that a mesh does.

The mesh pattern may be die-pressed, laser-cut, rolled, etc., onto the skin graft. If cut using a die or roller, the blades are thin and sharp so as to cut slits into the skin to create the mesh pattern. Furthermore, other methods to cut the pattern could be used.

The patterns will not provide the same extent of stretch that the parallel mesh provides in the direction perpendicular to the mesh lines.

As shown in each of FIGS. 2B and 2C of the alternating pattern, and FIGS. 5B and 5C of the herringbone pattern, these mesh patterns have been laser-cut in a prototype material.

These mesh patterns of the present disclosure may be applied to sheet products other than human dermis, which includes, but is not limited to, allograft amnion, allograft fascia, and synthetic surgical meshes.

In various embodiments, an after manufacture of a patterned surface and to provide complete a shelf-stable, packaged meshed ADM graft product. The pre-shaped, meshed ADM graft may be packaged along with two opposing pieces of backing material and sterile water in a sealed medical sterilization pouch, such as, for example, a Kapak pouch (manufactured by AMPAK Technology Inc. of Larchmont, NY), or further into a sealed, peelable medical sterilization pouch known as a “peel pouch” or a “chevron pouch.” The packaged ADM graft product may then be irradiated to a sterility assurance level (SAL) of 10⁻⁶ such that it may be stored at room temperature for up to two years. The packaged ADM graft product 170 may be labeled in any appropriate manner and may include information pertaining to the raw material, the shape, a use by date, special requirements, results of a visual inspection, and so on.

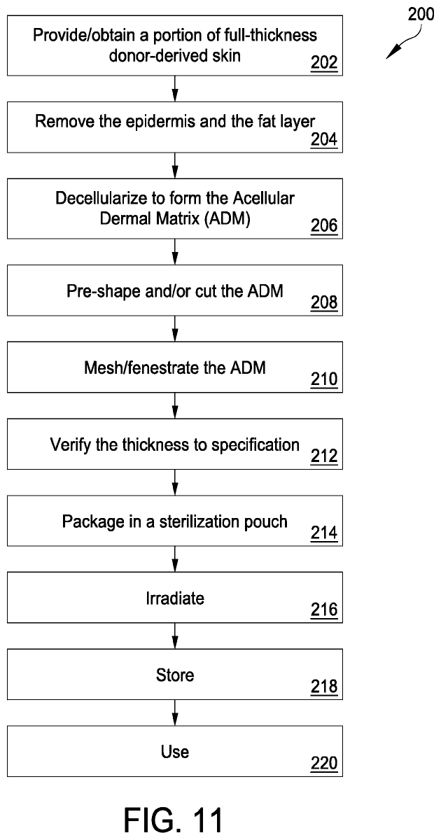

FIG. 11 provides a flowchart depicting an exemplary method (200) of manufacturing an embodiment of the pre-shaped, meshed ADM graft, and the packaged ADM product, discussed above. In this embodiment, the method may initiate with providing a portion of full-thickness donor-derived skin (202). Next, the epidermis layer and the fat layer adjacent to the dermis may be removed (204), and the dermal tissue may be decellularized according to a well-known or a proprietary decellularization process, resulting in the Acellular Dermal Matrix (ADM) (206). The ADM may then be shaped and/or cut into a pre-defined shape, such as the rectangular tissue portion or circular tissue portion or another appropriate shape, as necessary for an associated or pre-determined/assigned surgical procedure (208). The shaping may be accomplished using any appropriate scoring tool or another appropriate shaping tool, and the graft may be cut out with the cutting tool.

The ADM may also be meshed/fenestrated in the desired mesh pattern (e.g., 1:1 graft:space ratio, 2:1 graft:space ratio) using any appropriate skin mesher 140 (210). The meshing or fenestrating process (210) may occur before or after the ADM is shaped into the pre-defined shape. The resulting pre-shaped, meshed ADM graft 100 may then be verified for its thickness to specification (e.g., 1 mm-2 mm) (212) using a thickness gauge. In various embodiments, one or more antimicrobial agents may be added to the pre-shaped, meshed ADM graft 100 to aid in post-surgical infection prevention. The graft 100 may then be packaged (214) between opposing pieces of backing material 172 within sterile water inside a self-sealing medical sterilization pouch 174 and/or a peelable pouch 176 such as, for example, a Kapak peel-pouch, forming the pre-shaped, meshed ADM graft product 170. The packaged ADM graft product 170 may be irradiated to SAL 10⁻⁶ (216). After irradiation (216), the packaged, pre-shaped, meshed ADM graft product 170 may be stored up to two years (218) before it is used in a surgical procedure (220).

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

1. (canceled)

2. The packaged allograft implant of claim 9, wherein the mesh lines have a length of 1.0 mm to 3.48 mm.

3. The packaged allograft implant of claim 9, wherein the mesh lines have a length of 3.48 mm.

4. The packaged allograft implant of claim 9, wherein the mesh lines have a spacing apart from one another of 1.0 mm to 1.5 mm.

5. The packaged allograft implant of claim 9, wherein the mesh lines have a spacing apart from one another of 1.5 mm.

6-8. (canceled)

9. A packaged allograft implant configured for implantation in a human recipient, the packaged allograft implant comprising:
an acellular dermal matrix sheet having a top surface and a bottom surface in opposition to one another, a perimeter surrounding the top surface and the bottom surface, and a thickness extending between the top surface and the bottom surface; and
a mesh pattern extending across at least a portion of the top surface and the bottom surface of the acellular dermal matrix sheet, the mesh pattern providing through-holes extending between the top surface and the bottom surface of the acellular dermal matrix sheet, and the mesh pattern having a plurality of mesh lines extending in a first direction and a second direction, wherein the first direction and the second direction are orthogonal to one another so as to allow a given amount of stretch in each of the first direction and the second direction;
wherein the mesh pattern is a multiple quadrant pattern, with parallel mesh lines in opposing quadrants and perpendicular mesh lines in neighboring quadrants, and the multiple quadrant pattern contained in a circular shaped perimeter with the mesh lines.

10. A packaged allograft implant configured for implantation in a human recipient, the packaged allograft implant comprising:
an acellular dermal matrix sheet having a top surface and a bottom surface in opposition to one another, a perimeter surrounding the top surface and the bottom surface, and a thickness extending between the top surface and the bottom surface; and
a mesh pattern extending across at least a portion of the top surface and the bottom surface of the acellular dermal matrix sheet, the mesh pattern providing through-holes extending between the top surface and the bottom surface of the acellular dermal matrix sheet, and the mesh pattern having a plurality of mesh lines extending in a first direction and a second direction, wherein the first direction and the second direction are orthogonal to one another so as to allow a given amount of stretch in each of the first direction and the second direction;
wherein the mesh pattern is a row pattern formed by column of horizontal mesh lines followed by a column of vertical mesh lines, with the lines alternating for each column, with the mesh lines disposed on an acellular dermal matrix sheet forming an allograft for implantation in a human recipient.

11. (canceled)

12. A packaged allograft implant configured for implantation in a human recipient, the packaged allograft implant comprising:
an acellular dermal matrix sheet having a top surface and a bottom surface in opposition to one another, a perimeter surrounding the top surface and the bottom surface, and a thickness extending between the top surface and the bottom surface; and
a mesh pattern extending across at least a portion of the top surface and the bottom surface of the acellular dermal matrix sheet, the mesh pattern providing through-holes extending between the top surface and the bottom surface of the acellular dermal matrix sheet, and the mesh pattern having a plurality of mesh lines extending in a first direction and a second direction, wherein the first direction and the second direction are orthogonal to one another so as to allow a given amount of stretch in each of the first direction and the second direction;
wherein the mesh pattern is an alternating semi-circle mesh pattern with arcuate mesh lines positioned in an alternating configuration with one another, with the mesh lines disposed on an acellular dermal matrix sheet forming an allograft for implantation in a human recipient.

13-19. (canceled)

20. The packaged allograft implant of claim 10, wherein the mesh lines have a length of 1.0 mm to 3.48 mm.

21. The packaged allograft implant of claim 10, wherein the mesh lines have a length of 3.48 mm.

22. The packaged allograft implant of claim 10, wherein the mesh lines have a spacing apart from one another of 1.0 mm to 1.5 mm.

23. The packaged allograft implant of claim 10, wherein the mesh lines have a spacing apart from one another of 1.5 mm.

24. The packaged allograft implant of claim 12, wherein the mesh lines have a length of 1.0 mm to 3.48 mm.

25. The packaged allograft implant of claim 12, wherein the mesh lines have a length of 3.48 mm.

26. The packaged allograft implant of claim 12, wherein the mesh lines have a spacing apart from one another of 1.0 mm to 1.5 mm.

27. The packaged allograft implant of claim 12, wherein the mesh lines have a spacing apart from one another of 1.5 mm.
